(19) United States
(12) Patent Application Publication
(10) Pub. No.: US 2005/0191329 A1
(43) Pub. Date: Sep. 1, 2005

(54) Solid water-in-oil emulsion make-up composition

(76) Inventor: Toshiya Taniguchi, Kobe (JP)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/063,103

(22) Filed: Feb. 22, 2005

Related U.S. Application Data
(60) Provisional application No. 60/548,143, filed on Feb. 26, 2004.

(51) Int. Cl. A61K 7/06; A61K 7/11; A61K 7/00

(52) U.S. Cl. 424/401; 424/70.12

(57) ABSTRACT

Disclosed is a solid water-in-oil emulsified make-up composition comprising by weight: (a) from about 10% to about 25% of a powder component; the powder component comprising by weight of the powder component from about 50% to about 80% of a spherical powder having an average particle size of from about 0.1 μm to about 50 μm; the spherical powder comprising by weight of the entire composition: (i) from about 2% to about 20% of a spherical soft focus powder having a Total Luminous Transmittance (Tt) of from about 55 to about 90, a Diffuse Luminous Transmittance (Td) of from about 34 to about 81 and a Haze value {(Td/Tt)×100} of from about 62 to about 90; and (ii) from about 1% to about 10% of a spherical oil absorbing powder having an oil absorbency of at least about 100 ml/100 g; (b) a volatile silicone oil; (c) a non-volatile oil; (d) a solid wax; (e) a lipophilic surfactant having an HLB of less than about 8; and (f) water.

### CROSS REFERENCE TO RELATED APPLICATION

This Application claims the benefit of U.S. Provisional Application No. 60/548,143, filed Feb. 26, 2004.

### FIELD OF THE INVENTION

The present invention relates to a solid make-up composition in water-in-oil emulsion phase type. Specifically, the present invention relates to make-up compositions which have specified levels of components which provide the benefits of both a solid composition and a liquid composition, and also provide improved appearance. The compositions of the present invention are particularly useful for foundation products.

### BACKGROUND

A foundation composition can be applied to the face and other parts of the body to even skin tone and texture and to hide pores, imperfections, fine lines and the like. A foundation composition is also applied to moisturize the skin, to balance the oil level of the skin, and to provide protection against the adverse effects of sunlight, wind, and other environmental factors.

Foundation compositions are generally available in the form of liquid or cream suspensions, emulsions, gels, pressed powders or anhydrous oil and wax compositions. Emulsion-type foundations in the form of liquid are suitable in that they provide moisturizing effects by the water and water-soluble skin treatment agents incorporated. These liquid form foundations, however, are less convenient to use and carry for the consumer. On the other hand, solid foundations packaged in compacts are suitable for use by the consumer, however, are typically less efficient than liquid form foundations in terms of moisturizing the skin and coverage of the skin.

Foundation compositions in the form of solid, yet water-in-oil emulsion have been suggested. Such solid emulsion foundations aim to address the drawbacks of conventional liquid form foundations and solid foundations. These foundations can be filled in a wide variety of packaging, including compacts, and is increasing popularity among consumers. References which disclose such foundation compositions include Japanese patent publications A-2-88511, A-3-261707, A-7-267819, A-11-209243, U.S. Pat. No. 5,362,482, and PCT publication WO 01/91704.

Recently, consumers have become to seek foundation products that have the so-called “soft-focus effect”, namely, products with natural finish yet having good coverage for minimizing the appearance of skin troubles. Such an effect is achieved when two parameters are met. First, the contrast between lighted area of the skin and shaded area of the skin (such as pores and wrinkles) is minimized for reducing the appearance of the trouble areas. Second, the image of the skin is blurred for providing an overall natural finish. Various so-called “soft-focus pigments” are incorporated for providing such benefit. Incorporation of these pigments in solid emulsion foundations without deterioration of other characteristics has been a challenge, as the total amount of pigments that can be incorporated in such product form is limited.

Based on the foregoing, there is a need for a solid water-in-oil emulsion make-up composition which provides improved appearance to the skin, yet also provides good spreadability and moisturization to the skin and leaves the skin with a fresh and light feel.

None of the existing art provides all of the advantages and benefits of the present invention.

### SUMMARY OF THE INVENTION

The present invention is directed to a solid water-in-oil emulsified make-up composition comprising by weight:
(a) from about 10% to about 25% of a pigment component, the pigment component comprising by weight of the pigment component from about 50% to about 80% of a spherical pigment having an average particle size of from about 0.1 μm to about 50 μm; the spherical pigment comprising by weight of the entire composition:
   (i) from about 2% to about 20% of a spherical soft focus pigment having a Total Luminous Transmittance (Tt) of from about 55 to about 90, a Diffuse Luminous Transmittance (Td) of from about 34 to about 81, and a Haze value {(Td/Tt)×100} of from about 62 to about 90; and
   (ii) from about 1% to about 10% of a spherical oil absorbing pigment having an oil absorbency of at least about 100 ml/100 g;
(b) a volatile silicone oil;
(c) a non-volatile oil;
(d) a solid wax
(e) a lipophilic surfactant having an HLB of less than about 8; and
(f) water.

By formulating pigments of certain characteristics at an appropriate amount and ratio, a solid water-in-oil emulsion make-up composition which provides improved appearance to the skin, yet also provides good spreadability and moisturization to the skin and leaves the skin with a fresh and light feel is obtained.

These and other features, aspects, and advantages of the present invention will become evident to those skilled in the art from a reading of the present disclosure with the appended claims.

### DETAILED DESCRIPTION OF THE INVENTION

While the specification concludes with claims particularly pointing out and distinctly claiming the invention, it is believed that the present invention will be better understood from the following description.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

All ingredients such as actives and other ingredients useful herein may be categorized or described by their cosmetic and/or therapeutic benefit or their postulated mode of action. However, it is to be understood that the active and other ingredients useful herein can, in some instances, provide more than one cosmetic and/or therapeutic benefit or operate via more than one mode of action. Therefore, classifications herein are made for the sake of convenience and are not intended to limit an ingredient to the particularly stated application or applications listed.

Pigment Component

The composition of the present invention comprises from about 10% to about 25% of a pigment component. The pigments included in the pigment component herein are typically hydrophobic in nature, or hydrophobically treated. By keeping the level of pigment component low, the entire composition maintains flexibility to accommodate other components which provide spreadability, moisturization, and fresh and light feel. The pigment component herein comprises from about 50% to about 80% by weight of the pigment component of a spherical pigment, as detailed below.

The species and levels of the pigments other than spherical pigments are selected to provide, for example, shade, coverage, UV protection benefit, good wear performance, and stability in the composition.

Pigments other than spherical pigments that are useful for the pigment component herein are clay mineral powders such as talc, mica, sericite, silica, magnesium silicate, synthetic fluorphlogopite, calcium silicate, aluminum silicate, bentonite and montmorillonite; pearl pigments such as alumina, barium sulfate, calcium secondary phosphate, calcium carbonate, titanium oxide, finely divided titanium oxide, zirconium oxide, zinc oxide, hydroxy apatite, iron oxide, iron titanate, ultramarine blue, Prussian blue, chromium oxide, chromium hydroxide, cobalt oxide, cobalt titanate, titanium oxide coated mica; organic powders such as polyester, polyethylene, polystyrene, methyl methacrylate resin, cellulose, 12-nylon, 6-nylon, styrene-acrylic acid copolymers, polypropylene, vinyl chloride polymer, tetrafluoroethylene polymer, boron nitride, fish scale guanine, laked tar color dyes, and laked natural color dyes. Such pigments may be treated with a hydrophobical treatment agent, including: silicone such as Methicone, Dimethicone and perfluoroalkylsilane; fatty material such as stearic acid; metal soap such as aluminium dimyristate; aluminium hydrogenated tallow glutamate, hydrogenated lecithin, lauroyl lysine, aluminium salt of perfluoroalkyl phosphate, and mixtures thereof.

Spherical Pigment

From about 50% to about 80% of the pigment component herein is made of a spherical pigment having an average particle size of from about 0.1 μm to about 50 μm, preferably from about 1 μm to about 30 μm. Without being bound by theory, it is believed that the abundant amount of overall spherical pigments herein provides improved spreadability to the skin when the composition is applied.

The spherical pigments herein further comprise from about 2% to about 20% by weight of the entire composition of a spherical soft focus pigment, and from about 1% to about 10% by weight of the entire composition of a spherical oil absorbing pigment.

What is meant by spherical soft focus pigment is a pigment that is particularly effective in providing a soft focus effect to the composition, namely natural finish yet having good coverage for minimizing the appearance of skin troubles, when incorporated in the defined amount. Specifically, the soft focus pigment herein must meet two parameters for providing such an effect. First, both the Total Luminous Transmittance (Tt) and Diffuse Luminous Transmittance (Td) of the pigment are relatively high. The spherical soft focus pigments of the present invention have a Total Luminous Transmittance (Tt) of from about 55 to about 90 and a Diffuse Luminous Transmittance (Td) of from about 34 to about 81. Without being bound by theory, it is believed that, by having such high values, the spherical soft focus pigment exhibits a high transparency, thereby providing an overall natural finish. Second, the spherical soft focus pigments of the present invention have a relatively high Haze value {(Td/Tt)×100} of from about 62 to about 90. Without being bound by theory, it is believed that, by having such high Haze value, the contrast between lighted area of the skin and shaded area of the skin (such as pores and wrinkles) is minimized for reducing the appearance of the trouble areas. The use of the soft focus pigment herein having such high Total Luminous Transmittance (Tt) and Diffuse Luminous Transmittance (Td), and high Haze value {(Td/Tt)×100} is particularly effective for the present composition which contains a relatively low level of total pigment component.

Total Luminous Transmittance (Tt), Diffuse Luminous Transmittance (Td), and Haze value {(Td/Tt)×100} can be measured and calculated by the artisan by reference to ASTM D 1003-00 “Standard Test Method for Haze and Luminous Transmittance of Transparent Plastics”. Although the pigments herein are not plastics, the same principles of this specific standard test can be applied.

Spherical soft focus pigments useful herein include spherical alumina, such as those commercially available with tradename SA-Alumina Beads, available from Miyoshi Kasei Inc., having a Total Luminous Transmittance (Tt) of 62-72, Diffuse Luminous Transmittance (Td) of 45-55, and Haze value {(Td/Tt)×100} of 70-80.

What is meant by spherical oil absorbing pigment is a pigment that is particularly effective in absorbing oil, and thereby can be included in the present composition for absorbing excessive sebum from the skin. Specifically, the spherical oil absorbing pigment herein has an oil absorbency of at least about 100 ml/100 g, preferably at least about 200 ml/100 g. Oil absorbency is a unit well known to the artisan, and which can be measured via: JIS K5101 No. 21 “Test Method for Oil Absorbency Level”.

Spherical oil absorbing pigments useful herein include spherical silica, spherical silicone elastomer, and methyl methacrylate copolymer. Commercially available spherical oil absorbing pigments useful herein include spherical silica with tradename SI-SILDEX H-52 available from Miyoshi Kasei, Inc. having an oil absorbency of more than 200 ml/100 g, vinyl dimethicone/methicone silsesquioxane crosspolymer with tradename KSP-100 and KSP-101 available from ShinEtsu Chemical having an oil absorbency of more than 200 ml/100 g, hardened polyorgano siloxane elastomers with tradename TREFIL E-506C available from Dow Corning having an oil absorbency of more than 100 ml/100 g, and methyl methacrylate copolymer with tradename SA-GMP-0820 available from GANZ Chemical and surface treated by Miyoshi Kasei, Inc. having an oil absorbency of more than 100 ml/100 g.

Spherical pigments other than the soft focus pigments and oil absorbing pigments may also be used. Unlimited examples of materials useful for making the spherical powders are; polyacrylates, silicates, sulfates, metal dioxides, carbonates, celluloses, polyalkylenes, vinyl acetates, polystyrenes, polyamides, acrylic acid ethers, silicones, and mixtures and complexes thereof. Specifically, materials useful herein include polyacrylates such as nylon, silicates such as calcium silicate, magnesium silicate, barium silicate, aluminium silicate and silica beads; metal dioxides such as titanium dioxide and aluminium hydroxide; carbonates such as calcium carbonate, magnesium carbonate; celluloses; polyalkylenes such as polyethylene, and polypropylene; vinyl acetates; polystyrenes; polyamides; acrylic acid ethers such as acrylic acid methyl ether and acrylic acid ethyl ether; polyvinyl pyrrolidones; and silicones such as polyorganosilsesquioxane resin.

Commercially available spherical powders highly useful herein include Nylon-12 with tradename NYLON POWDER series available from Toray.

Volatile Silicone Oil

The composition of the present invention comprises a volatile silicone oil. Preferably, the amount of the volatile silicone oil is controlled so that the composition comprises from about 20% to about 50% of the volatile silicone oil, and the total of the volatile silicone oil and water is more than about 50% of the entire composition. Without being bound by theory, the species and levels of the volatile silicone oil herein is believed to provide improved refreshing and light feeling to the skin, without necessarily leaving a dried feeling to the skin.

The volatile silicone oil useful herein are selected from those having a boiling point of from about 60 to about 260° C., preferably those having from 2 to 7 silicon atoms. The volatile silicone oils useful herein include polyalkyl or polyaryl siloxanes with the following structure (I): 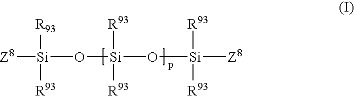
wherein R⁹³ is independently alkyl or aryl, and p is an integer from about 0 to about 5. Z⁸ represents groups which block the ends of the silicone chains. Preferably, R⁹³ groups include methyl, ethyl, propyl, phenyl, methylphenyl and phenylmethyl, Z⁸ groups include hydroxy, methyl, methoxy, ethoxy, propoxy, and aryloxy. More preferably, R⁹³ groups and Z⁸ groups are methyl groups. The preferred volatile silicone compounds are hexamethyldisiloxane, octamethyltrisiloxane, decamethyltetrasiloxane, hexadecamethylheptasiloxane. Commercially available volatile silicone compounds useful herein include octamethyltrisiloxane with tradename SH200C-1cs, decamethyltetrasiloxane with tradename SH200C-1.5cs, hexadecamethylheptasiloxane with tradename SH200C-2cs, all available from Dow Corning.

The volatile silicone oils useful herein also include a cyclic silicone compound having the formula: 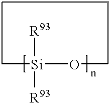
wherein R⁹³ is independently alkyl or aryl, and n is an integer of from 3 to 7.

Preferably, R⁹³ groups include methyl, ethyl, propyl, phenyl, methylphenyl and phenylmethyl. More preferably, R⁹³ groups are methyl groups. The preferred volatile silicone compounds are octamethylcyclotetrasiloxane, decamethylcyclopentasiloxane, tetradecamethylcyclohexasiloxane. Commercially available volatile silicone compounds useful herein include octamethylcyclotetrasiloxane with tradename SH244, decamethylcyclopentasiloxane with tradename DC245 and SH245, and dodeamethylcyclohexasiloxane with tradename DC246; all available from Dow Corning.

Non-Volatile Oil

The composition of the present invention comprises a non-volatile oil, preferably by weight of the entire composition at from about 0.5% to about 10%. Without being bound by theory, the species and levels of the non-volatile oil herein is believed to provide improved smoothness to the skin, and also alleviate dry feeling of the skin.

Non-volatile oils useful herein are, for example, tridecyl isononanoate, isostearyl isostearate, isocetyl isosteatrate, isopropyl isostearate, isodecyl isonoanoate, cetyl octanoate, isononyl isononanoate, diisopropyl myristate, isocetyl myristate, isotridecyl myristate, isopropyl myristate, isostearyl palmitate, isocetyl palmitate, isodecyl palmitate, isopropyl palmitate, octyl palmitate, caprylic/capric acid triglyceride, glyceryl tri-2-ethylhexanoate, neopentyl glycol di(2-ethyl hexanoate), diisopropyl dimerate, tocopherol, tocopherol acetate, avocado oil, camellia oil, turtle oil, macadamia nut oil, corn oil, mink oil, olive oil, rapeseed oil, eggyolk oil, sesame oil, persic oil, wheat germ oil, pasanqua oil, castor oil, linseed oil, safflower oil, cotton seed oil, perillic oil, soybean oil, peanut oil, tea seed oil, kaya oil, rice bran oil, china paulownia oil, Japanese paulownia oil, jojoba oil, rice germ oil, glycerol trioctanate, glycerol triisopalmiatate, trimethylolpropane triisostearate, isopropyl myristate, glycerol tri-2-ethylhexanoate, pentaerythritol tetra-2-ethylhexanoate, lanolin, liquid lanolin, liquid paraffin, squalane, vaseline, and mixtures thereof. Commercially available oils include, for example, tridecyl isononanoate with tradename Crodamol TN available from Croda, Hexalan available from Nisshin Seiyu, and tocopherol acetates available from Eisai.

Non-volatile oils useful herein also include polyalkyl or polyaryl siloxanes with the following structure (I) 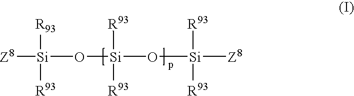
wherein R⁹³ is alkyl or aryl, and p is an integer from about 7 to about 8,000. Z⁸ represents groups which block the ends of the silicone chains. The alkyl or aryl groups substituted on the siloxane chain (R⁹³) or at the ends of the siloxane chains Z⁸ can have any structure as long as the resulting silicone remains fluid at room temperature, is dispersible, is neither irritating, toxic nor otherwise harmful when applied to the skin, is compatible with the other components of the composition, and is chemically stable under normal use and storage conditions. Suitable Z⁸ groups include hydroxy, methyl, methoxy, ethoxy, propoxy, and aryloxy. The two R⁹³ groups on the silicon atom may represent the same group or different groups. Preferably, the two R⁹³ groups represent the same group. Suitable R⁹³ groups include methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known as dimethicone, is especially preferred. The polyalkylsiloxanes that can be used include, for example, polydimethylsiloxanes. These silicone compounds are available, for example, from the General Electric Company in their Viscasil® and SF 96 series, and from Dow Corning in their Dow Corning 200 series.

Polyalkylaryl siloxane fluids can also be used and include, for example, polymethylphenylsiloxanes. These siloxanes are available, for example, from the General Electric Company as SF 1075 methyl phenyl fluid or from Dow Corning as 556 Cosmetic Grade Fluid.

Non-volatile oils also useful herein are the various grades of mineral oils. Mineral oils are liquid mixtures of hydrocarbons that are obtained from petroleum. Specific examples of suitable hydrocarbons include paraffin oil, mineral oil, dodecane, isododecane, hexadecane, isohexadecane, eicosene, isoeicosene, tridecane, tetradecane, polybutene, polyisobutene, and mixtures thereof.

Solid Wax

The composition of the present invention comprises a solid wax, preferably by weight of the entire composition at from about 1% to about 5%. Without being bound by theory, the species and levels of the solid wax herein is believed to provide consistency to the composition and coverage to the skin, while not negatively contributing to the spreadability upon application to the skin, and fresh and light feel of the skin.

The solid waxes useful herein are paraffin wax, microcrystalline wax, ozokerite was, ceresin wax, carnauba wax, candellila wax, eicosanyl behenate, and mixtures thereof. A mixture of waxes is preferably used.

Commercially available solid waxes useful herein include: Candelilla wax NC-1630 available from Cerarica Noda, Ozokerite wax SP-1021 available from Strahl & Pitsh, and Eicosanyl behenate available from Cas Chemical.

Lipophilic Surfactant

The composition of the present invention comprises a lipophilic surfactant, preferably by weight of the entire composition at from about 1% to about 5%. The lipophilic surfactant herein has an HLB value of less than about 8.

The HLB value is a theoretical index value which describes the hydrophilicity-hydrophobicity balance of a specific compound. Generally, it is recognized that the HLB index ranges from 0 (very hydrophobic) to 40 (very hydrophilic). The HLB value of the lipophilic surfactants may be found in tables and charts known in the art, or may be calculated with the following general equation: HLB=7+(hydrophobic group values)+(hydrophilic group values). The HLB and methods for calculating the HLB of a compound are explained in detail in Surfactant Science Series, Vol. 1: Nonionic Surfactants”, pp 606-13, M. J. Schick (Marcel Dekker Inc., New York, 1966).

Without being bound by theory, the species and levels of the lipophilic surfactant herein are believed to provide a stable water-in-oil emulsion in view of the other components of the present invention.

The lipophilic surfactant can be an ester-type surfactant. Ester-type surfactants useful herein include: sorbitan monoisostearate, sorbitan diisostearate, sorbitan sesquiisostearate, sorbitan monooleate, sorbitan dioleate, sorbitan sesquioleate, glyceryl monoisostearate, glyceryl diiostearate, glyceryl sesquiisostearate, glyceryl monooleate, glyceryl dioleate, glyceryl sesquioleate, diglyceryl diisostearate, diglyceryl dioleate, diglycerin monoisostearyl ether, diglycerin diisostearyl ether, and mixtures thereof.

Commercially available ester-type surfactants are, for example, sorbitan isostearate having a tradename Crill 6 available from Croda, and sorbitan sesquioleate with tradename Arlacel 83 available from Kao Atras.

The lipophilic surfactant can be a silicone-type surfactant. Silicone-type surfactants useful herein are (i), (ii), and (iii) as shown below, and mixtures thereof.
(i) dimethicone copolyols having the formulation: 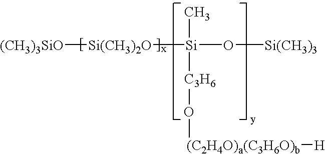
   wherein x is an integer from 5 to 100, y is an integer from 1 to 50, a is zero or greater, b is zero or greater, the average sum of a+b being 1-100.
(ii) dimethicone copolyols having the formulation: 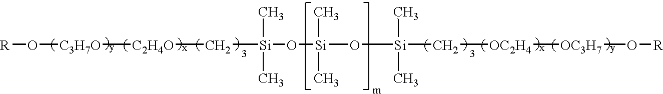
   wherein R is selected from the group consisting of hydrogen, methyl, and combinations thereof, m is an integer from 5 to 100, x is independently zero or greater, y is independently zero or greater, the sum of x+y being 1-100.
(iii) branched polyether-polydiorganosiloxane emulsifiers herein having the formulation: 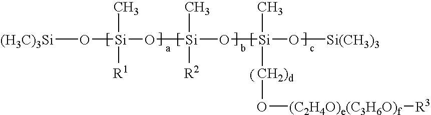
   wherein R¹ is an alkyl group having from about 1 to about 20 carbons; R² is 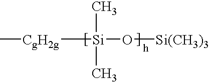
   wherein g is from about 1 to about 5, and h is from about 5 to about 20; R³ is H or an alkyl group having from about 1 to about 5 carbons; e is from about 5 to about 20; f is from about 0 to about 10; a is from about 20 to about 100; b is from about 1 to about 15; c is from about 1 to about 15; and d is from about 1 to about 5.

Commercially available silicone-type surfactants are, for example, dimethicone copolyols DC5225C, BY22-012, BY22-008, SH3746M, SH3771M, SH3772M, SH3773M, SH3775M, SH3748, SH3749, and DC5200, all available from Dow Corning, and branched polyether-polydiorganosiloxane emulsifiers such as PEG-9 polydimethylsiloxyethyl Dimethicone, having an HLB of about 4 and a molecular weight of about 6,000 having a tradename KF 6028 available from ShinEtsu Chemical.

In a preferred embodiment, the lipophilic surfactant is a mixture of at least one ester-type surfactant and at least one silicone-type surfactant to provide a stable emulsion for the other essential components of the present invention.

Water

The composition of the present invention comprises water in an amount sufficient to provide a discontinuous aqueous phase, preferably an amount such that the total of the volatile silicone oil and water is more than about 50% of the entire composition. More preferably, the present composition comprises from about 10% to about 40% of water. Without being bound by theory, the amount of water herein is believed to provide improved refreshing and light feeling to the skin, without necessarily leaving a dried feeling to the skin. Further, this amount of water allows the inclusion of optional water-soluble skin active agents as described below.

In the present invention, deionized water is typically used. Water from natural sources including mineral cations can also be used, depending on the desired characteristic of the product.

Humectant

The composition of the present invention may further comprise a humectant by weight of the entire composition at from about 1% to about 15%, preferably 2% to about 7%.

The humectants herein are selected from the group consisting of polyhydric alcohols, water soluble alkoxylated nonionic polymers, and mixtures thereof. Polyhydric alcohols useful herein include glycerin, propylene glycol, 1,3-butylene glycol, dipropylene glycol, diglycerin, sodium hyaluronate, and mixtures thereof.

Commercially available humectants herein include: glycerin available from Asahi Denka; propylene glycol with tradename LEXOL PG-865/855 available from Inolex, 1,2-PROPYLENE GLYCOL USP available from BASF; 1,3-butylene glycol available from Kyowa Hakko Kogyo; dipropylene glycol with the same tradename available from BASF; diglycerin with tradename DIGLYCEROL available from Solvay GmbH; sodium hyaluronate with tradenames ACTIMOIST available from Active Organics, AVIAN SODIUM HYALURONATE series available from Intergen, HYALURONIC ACID Na available from Ichimaru Pharcos.

Film Forming Polymer

The compositions of the present invention may comprise a film forming polymer, for imparting wear and/or transfer resistant properties. When included, such materials are typically used in an amount of from about 0.5% to about 20% preferably from about 0.5% to about 10% by weight, more preferably from about 1% to about 8% by weight of the composition. Preferred polymers form a non-tacky film which is removable with water used with cleansers such as soap.

Examples of suitable film forming polymeric materials include:
a) sulfopolyester resins, such as AQ sulfopolyester resins, such as AQ29D, AQ35S, AQ38D, AQ38S, AQ48S, and AQ55S (available from Eastman Chemicals);
b) polyvinylacetate/polyvinyl alcohol polymers, such as Vinex resins available from Air Products, including Vinex 2034, Vinex 2144, and Vinex 2019;
c) acrylic resins, including water dispersible acrylic resins available from National Starch under the trade name “Dermacryl”, including Dermacryl LT;
d) polyvinylpyrrolidones (PVP), including Luviskol K17, K30 and K90 (available from BASF), water soluble copolymers of PVP, including PVPNA S-630 and W-735 and PVP/dimethylaminoethylmethacrylate Copolymers such as Copolymer 845 and Copolymer 937 available from ISP, as well as other PVP polymers disclosed by E. S. Barabas in the Encyclopedia of Polymer Science and Engineering, 2 Ed. Vol. 17 pp. 198-257;
e) high molecular weight silicones such as dimethicone and organic-substituted dimethicones, especially those with viscosities of greater than about 50,000 mPas;
f) high molecular weight hydrocarbon polymers with viscosities of greater than about 50,000 mPas;
g) organosiloxanes, including organosiloxane resins, fluid diorganopolysiloxane polymers and silicone ester waxes.

Examples of these polymers and cosmetic compositions containing them are found in PCT publication Nos. WO96/33689, published Oct. 31, 1996; WO97/17058, published May 15, 1997; and U.S. Pat. No. 5,505,937 issued to Castrogiovanni et al. Apr. 9, 1996, all incorporated herein by reference. Additional film forming polymers suitable for use herein include the water-insoluble polymer materials in aqueous emulsion and water soluble film forming polymers described in PCT publication No. WO98/18431, published May 7, 1998, incorporated herein by reference. Examples of high molecular weight hydrocarbon polymers with viscosities of greater than about 50,000 mPas include polybutene, polybutene terephthalate, polydecene, polycyclopentadiene, and similar linear and branched high molecular weight hydrocarbons.

Preferred film forming polymers include organosiloxane resins comprising combinations of R₃SiO_{1/2} “M” units, R₂SiO “D” units, RSiO_{3/2} “T” units, SiO₂ “Q” units in ratios to each other that satisfy the relationship RnSiO_{(4-n)/2} where n is a value between 1.0 and 1.50 and R is a methyl group. Note that a small amount, up to 5%, of silanol or alkoxy functionality may also be present in the resin structure as a result of processing. The organosiloxane resins must be solid at about 25° C. and have a molecular weight range of from about 1,000 to about 10,000 grams/mole. The resin is soluble in organic solvents such as toluene, xylene, isoparaffins, and cyclosiloxanes or the volatile carrier, indicating that the resin is not sufficiently crosslinked such that the resin is insoluble in the volatile carrier. Particularly preferred are resins comprising repeating monofunctional or R₃SiO_{1/2} “M” units and the quadrofunctional or SiO₂ “Q” units, otherwise known as “MQ” resins as disclosed in U.S. Pat. No. 5,330,747, Krzysik, issued Jul. 19, 1994, incorporated herein by reference. In the present invention the ratio of the “M” to “Q” functional units is preferably about 0.7 and the value of n is 1.2. Organosiloxane resins such as these are commercially available such as Wacker 803 and 804 available from Wacker Silicones Corporation of Adrian Mich., KP545 from Shin-Etsu Chemical and G. E. 1170-002 from the General Electric Company.

Skin Active Agent

The compositions of the present invention may comprise a safe and effective amount of a skin active agent. The term “skin active agent” as used herein, means an active ingredient which provides a cosmetic and/or therapeutic effect to the area of application on the skin, hair, or nails. The skin active agents useful herein include skin lightening agents, anti-acne agents, emollients, non-steroidal anti-inflammatory agents, topical anaesthetics, artificial tanning agents, antiseptics, anti-microbial and anti-fungal actives, skin soothing agents, sunscreening agents, skin barrier repair agents, anti-wrinkle agents, anti-skin atrophy actives, lipids, sebum inhibitors, sebum inhibitors, skin sensates, protease inhibitors, skin tightening agents, anti-itch agents, hair growth inhibitors, desquamation enzyme enhancers, anti-glycation agents, and mixtures thereof. When included, the present composition comprises from about 0.001% to about 30%, preferably from about 0.001% to about 10% of at least one skin active agent.

The type and amount of skin active agents are selected so that the inclusion of a specific agent does not affect the stability of the composition. For example, hydrophilic agents may be incorporated in an amount soluble in the aqueous phase, while lipophilic agents may be incorporated in an amount soluble in the oil phase.

Skin lightening agents useful herein refer to active ingredients that improve hyperpigmentation as compared to pre-treatment. Useful skin lightening agents herein include ascorbic acid compounds, vitamin B₃ compounds, azelaic acid, butyl hydroxyanisole, gallic acid and its derivatives, glycyrrhizinic acid, hydroquinone, kojic acid, arbutin, mulberry extract, and mixtures thereof. Use of combinations of skin lightening agents is believed to be advantageous in that they may provide skin lightening benefit through different mechanisms.

Ascorbic acid compounds useful herein include, ascorbic acid per se in the L-form, ascorbic acid salt, and derivatives thereof. Ascorbic acid salts useful herein include, sodium, potassium, lithium, calcium, magnesium, barium, ammonium and protamine salts. Ascorbic acid derivatives useful herein include, for example, esters of ascorbic acid, and ester salts of ascorbic acid. Particularly preferred ascorbic acid compounds include 2-o-D-glucopyranosyl-L-ascorbic acid, which is an ester of ascorbic acid and glucose and usually referred to as L-ascorbic acid 2-glucoside or ascorbyl glucoside, and its metal salts, and L-ascorbic acid phosphate ester salts such as sodium ascorbyl phosphate, potassium ascorbyl phosphate, magnesium ascorbyl phosphate, and calcium ascorbyl phosphate. Commercially available ascorbic compounds include magnesium ascorbyl phosphate available from Showa Denko, 2-o-D-glucopyranosyl-L-ascorbic acid available from Hayashibara and sodium L-ascorbyl phosphate with tradename STAY C available from Roche.

Vitamin B₃ compounds useful herein include, for example, those having the formula:

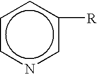

wherein R is —CONH₂ (e.g., niacinamide) or —CH₂OH (e.g., nicotinyl alcohol); derivatives thereof; and salts thereof. Exemplary derivatives of the foregoing vitamin B₃ compounds include nicotinic acid esters, including non-vasodilating esters of nicotinic acid, nicotinyl amino acids, nicotinyl alcohol esters of carboxylic acids, nicotinic acid N-oxide and niacinamide N-oxide. Preferred vitamin B₃ compounds are niacinamide and tocopherol nicotinate, and more preferred is niacinamide. In a preferred embodiment, the vitamin B₃ compound contains a limited amount of the salt form and is more preferably substantially free of salts of a vitamin B₃ compound. Preferably the vitamin B₃ compound contains less than about 50% of such salt, and is more preferably essentially free of the salt form. Commercially available vitamin B₃ compounds that are highly useful herein include niacinamide USP available from Reilly.

Other hydrophobic skin lightening agents useful herein include ascorbic acid derivatives such as ascorbyl tetraisopalmitate (for example, VC-IP available from Nikko Chemical), ascorbyl palmitate (for example available from Roche Vitamins), ascorbyl dipalmitate (for example, NIKKOL CP available from Nikko Chemical); undecylenoyl phenyl alanine (for example, SEPIWHITE MSH available from Seppic); octadecenedioic acid (for example, ARLATONE DIOIC DCA available from Uniquema); oenothera biennis sead extract, and pyrus malus (apple) fruit extract, and mixtures thereof.

Other skin active agents useful herein include those selected from the group consisting of panthenol, tocopheryl nicotinate, benzoyl peroxide, 3-hydroxy benzoic acid, flavonoids (e.g., flavanone, chalcone), farnesol, phytantriol, glycolic acid, lactic acid, 4-hydroxy benzoic acid, acetyl salicylic acid, 2-hydroxybutanoic acid, 2-hydroxypentanoic acid, 2-hydroxyhexanoic acid, cis-retinoic acid, trans-retinoic acid, retinol, retinyl esters (e.g., retinyl propionate), phytic acid, N-acetyl-L-cysteine, lipoic acid, tocopherol and its esters (e.g., tocopheryl acetate), azelaic acid, arachidonic acid, tetracycline, ibuprofen, naproxen, ketoprofen, hydrocortisone, acetominophen, resorcinol, phenoxyethanol, phenoxypropanol, phenoxyisopropanol, 2,4,4′-trichloro-2′-hydroxy diphenyl ether, 3,4,4′-trichlorocarbanilide, octopirox, lidocaine hydrochloride, clotrimazole, miconazole, ketoconazole, neomycin sulfate, theophylline, and mixtures thereof.

UV Absorbing Agent

The compositions of the present invention may comprise a safe and effective amount of a UV absorbing agent. A wide variety of conventional UV protecting agent are suitable for use herein, such as those decribed in U.S. Pat. No. 5,087,445, Haffey et al, issued Feb. 11, 1992; U.S. Pat. No. 5,073,372, Turner et al, issued Dec. 17, 1991; U.S. Pat. No. 5,073,371, Turner et al., issued Dec. 17, 1991; and Segarin, et al, at Chapter VIII, pages 189 et seq., of Cosmetics Science and Technology (1972). When included, the present composition comprises from about 0.5% to about 20%, preferably from about 1% to about 15% of a UV absorbing agent.

UV absorbing agents useful herein are, for example, 2-ethylhexyl-p-methoxycinnamate (commercially available as PARSOL MCX), butylmethoxydibenzoyl-methane, 2-hydroxy-4-methoxybenzo-phenone, 2-phenylbenzimidazole-5-sulfonic acid, octyldimethyl-p-aminobenzoic acid, octocrylene, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, Eusolex™ 6300, Octocrylene, Avobenzone (commercially available as Parsol 1789), and mixtures thereof.

Additional Components

The compositions hereof may further contain additional components such as are conventionally used in topical products, e.g., for providing aesthetic or functional benefit to the composition or skin, such as sensory benefits relating to appearance, smell, or feel, therapeutic benefits, or prophylactic benefits (it is to be understood that the above-described required materials may themselves provide such benefits).

The CTFA Cosmetic Ingredient Handbook, Second Edition (1992) describes a wide variety of nonlimiting cosmetic and pharmaceutical ingredients commonly used in the industry, which are suitable for use in the topical compositions of the present invention. Such other materials may be dissolved or dispersed in the composition, depending on the relative solubilities of the components of the composition.

Examples of suitable topical ingredient classes include: anti-cellulite agents, antioxidants, radical scavengers, chelating agents, vitamins and derivatives thereof, abrasives, other oil absorbents, astringents, dyes, essential oils, fragrance, structuring agents, emulsifiers, solubilizing agents, anti-caking agents, antifoaming agents, binders, buffering agents, bulking agents, denaturants, pH adjusters, propellants, reducing agents, sequestrants, cosmetic biocides, and preservatives.

Preparation of the Composition

The composition of the present invention may be made by a method well known in the art. In a suitable process, the composition is made by the steps of:
1) dissolving the volatile silicone oil, non-volatile oil, solid wax, lipophilic surfactant, slurry of pigments dispersed in oil, and any other hydrophobic material in liquid form at ambient temperature in a sealed tank, to make a lipophilic mixture;
2) adding the remaining pigments and powders into such lipophilic mixture and dispersing with a homogenizer at about 20-30° C.;
3) separate from 1) and 2), heating and dissolving in water, humectants and any other hydrophilic material at about 75-80° C., and then cooling to about 20-30° C.;
4) adding the product of step 3) to the product of step 2) to effect an emulsification;
5) heating and adding to the product of step 4) solid wax and any remaining hydrophobic material at about 80-85° C.; and
6) cooling the finally obtained emulsion to a temperature of about 60-80° C.
   The obtained composition, which is still fluid at such temperature, is filled in an air-tight container and allowed to cool to room temperature typically using a cooling unit. The obtained composition is solid at ambient temperature, and thus can be poured into such container and left to solidify. The air-tight container is typically in a package form of a compact.

### EXAMPLES

The following examples further describe and demonstrate the preferred embodiments within the scope of the present invention. The examples are given solely for the purpose of illustration, and are not to be construed as limitations of the present invention since many variations thereof are possible without departing from its spirit and scope.

### Examples 1-6

The following compositions are formed by the process described herein:

| No. | Components | Ex. 1 | Ex. 2 | Ex. 3 | Ex. 4 | Ex. 5 | Ex. 6 |
|---|---|---|---|---|---|---|---|
| 1 | Cyclopentasiloxane *1 | 33.2 | 30.2 | 30.2 | 27.2 | 34.2 | 33.2 |
| 2 | PEG-9 Polydimethylsiloxyethyl | 1 | 1 | 1 | 1 | 1 | 1 |
| | Dimethicone *2 | | | | | | |
| 3 | Dimethicone and Dimethicone/Vinyl | | | | 3 | | |
| | Dimethicone Crosspolymer *3 | | | | | | |
| 4 | Ethylhexyl Methoxycinnamate and BHT *4 | 5 | 5 | 5 | 5 | | 5 |
| 5 | Tocopheryl Acetate *5 | 0.5 | 0.5 | 0.5 | 0.5 | 0.5 | 0.5 |
| 6 | Isotridecyl Isononanoate *6 | 1 | 1 | 1 | 1 | 1 | 1 |
| 7 | Sorbitan Monoisostearate *7 | 1.5 | 1.5 | 1.5 | 1.5 | 1.5 | 1.5 |
| 8 | Slurry of Iron oxide and | 1.3 | 1.3 | 1.3 | 1.3 | 1.3 | 1.3 |
| | Cyclopentasiloxane and Dimethicone and | | | | | | |
| | Disodium Hydrogenated Glutamate *8 | | | | | | |
| 9 | Titanium Dioxide and Methicone *9 | | | | | 3 | |
| 10 | Titanium Dioxide and Methicone *10 | 7 | 5 | 4 | 5 | 3 | 7 |
| 11 | Alumina and Dimethicone *11 | 3 | 7 | 12 | 7 | 7 | 3 |
| 12 | Silica and Methicone *12 | 3 | 3 | 4 | 5 | 3 | 3 |
| 13 | Vinyl Dimethicone/Methicone | 5 | 3 | 3 | 1 | 3 | 5 |
| | Silsesquioxane Crosspolymer *13 | | | | | | |
| 14 | Alumina and Titanium Dioxide and | | 3 | | | | |
| | Methicone *14 | | | | | | |
| 15 | Titanium Dioxide and Dimethicone and | | | | | 3 | |
| | Aluminium Hydroxide and Stearic | | | | | | |
| | Acid *15 | | | | | | |
| 16 | Mica and Zinc Oxide and Methicone and | | | | 3 | | |
| | Hydroxyapatite *16 | | | | | | |
| 17 | Mica and Methicone *17 | 2 | 2 | | 2 | 2 | 2 |
| 18 | Water | 25 | 25 | 25 | 25 | 25 | 25 |
| 19 | Polyvinylpyrrolidone *18 | | | | | | 1 |
| 19 | Niacinamide *19 | 2 | 2 | 2 | 2 | 2 | 2 |
| 20 | Preservative | 0.45 | 0.45 | 0.45 | 0.45 | 0.45 | 0.45 |
| 21 | Panthenol *20 | 0.25 | 0.25 | 0.25 | 0.25 | 0.25 | 0.25 |
| 22 | Glycerin *21 | | | | | 5 | |
| 23 | Butylene Glycol *22 | 5 | 5 | 5 | 5 | | 5 |
| 24 | Candelilla Wax *23 | 2 | 2 | 2 | 2 | 2 | 2 |
| 25 | Ceresin *24 | 1.8 | 1.8 | 1.8 | 1.8 | 1.8 | 1.8 |

Definitions of Components
*1 Cyclomethicone: SH245 available from Dow Corning
*2 PEG-9 Polydimethylsiloxyethyl Dimethicone: KF-6028 available from Shinetsu Silicone
*3 Dimethicone and Dimethicone/Vinyl Dimethicone Cross Polymer: KSG-16 available from Shinetsu Silicone
*4 Ethylhexyl Methoxycinnamate and BHT: PARSOL MCX available from Roche
*5 Tocopheryl Acetate: DL-a-Tocopheryl Acetate available from Eisai
*6 Isotridecyl Isononanoate: Crodamol TN available from Croda
*7 Sorbitan Monoisostearate: Crill 6 available from Croda
*8 Slurry of Iron Oxide and Cyclopentasiloxane and Dimethicone and Disodium Hydrogenated Glutamate: SA/NAI-Y-10/D5 (70%), SA/NAI-R-10/D5 (65%) and SA/NAI-B-10/D5 (75%) available from Miyoshi Kasei
*9 Titanium Dioxide and Methicone: SI FTL-300 available from Miyoshi Kasei
*10 Titanium Dioxide and Methicone: SI Titanium Dioxide IS available from Miyoshi Kasei
*11 Alumina and Dimethicone: SA-Alumina Beads available from Miyoshi Kasei having a Total Luminous Transmittance (Tt) of 62-72, Diffuse Luminous Transmittance (Td) of 45-55, and Haze value {(Td/Tt) × 100} of 70-80
*12 Silica and Methicone: SI-SILDEX H-52 available from Asahi Glass Company Co., Ltd. and surface treated by Miyoshi Kasei, having an oil absorbency of more than 200 ml/100 g
*13 Vinyl Dimethicone/Methicone Silsesquioxane Crosspolymer: KSP-100 available from Shinetsu Silicone having an oil absorbency of more than 200 ml/100 g
*14 Alumina and Titanium Dioxide and Methicone: SI-LTSG30AFLAKEH(5%)LHC available from Nippon Sheet Glass Co., Ltd. and surface treated by Miyoshi Kasei
*15 Titanium Dioxide and Dimethicone and Aluminum Hydroxide and Stearic acid: SAST-UFTR-Z available from Miyoshi Kasei
*16 Mica and Zinc Oxide and Methicone and Hydroxyapatite: SI-PLV-20 available from Miyoshi Kasei
*17 Mica and Methicone: SI Mica available from Miyoshi Kasei
*18 Polyvinylpyrrolidone: PVP K-30 available from BASF
*19 Niacinamide: Niacinamide available from Reilly Industries Inc.
*20 Panthenol: DL-Panthenol available from Alps Pharmaceutical Ind.
*21 Glycerin: Glycerin USP available from Asahi Denka
*22 Butylene Glycol: 1,3 Butylene Glycol available from Kyowa Hakko Kogyo
*23 Candelilla wax: Candelilla wax NC-1630 available from Cerarica Noda
*24 Ceresin: Ozokerite wax SP-1021 available from Strahl & Pitsh

Method of Preparation

The compositions of Examples 1-6 are prepared as follows.

1) Component numbers 1 through 8 are mixed with suitable mixer until homogeneous to make a lipophilic mixture.
2) Component numbers 9 through 17 are mixed with suitable mixer until homogeneous to make a powder mixture. The powder mixture is pulverized using a pulverizer, and added into the lipophilic mixture with suitable a mixer until homogeneous.
3) Component numbers 18 through 23 are dissolved using a suitable mixer, and then added into the product of step 2) to effect emulsification at room temperature using a homogenizer.
4) Component number 24 and 25 are added into the product of step 3) and heated to dissolve at 85° C. in a sealed tank.
5) The finally obtained product is filled in an air-tight container and allowed to cool to room temperature using a cooling unit.

These embodiments represented by the previous examples have many advantages when applied to the skin as foundation products. For example, they can provide improved natural finish to the skin without compromise to good skin coverage, and further provide improved spreadability and moisturization to the skin, and leaves the skin with a fresh and light feel.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A solid water-in-oil emulsified make-up composition comprising by weight:
(a) from about 10% to about 25% of a powder component; the powder component comprising by weight of the powder component from about 50% to about 80% of a spherical powder having an average particle size of from about 0.1 μm to about 50 μm; the spherical powder comprising by weight of the entire composition:
(i) from about 2% to about 20% of a spherical soft focus powder having a Total Luminous Transmittance (Tt) of from about 55 to about 90, a Diffuse Luminous Transmittance (Td) of from about 34 to about 81 and a Haze value {(Td/Tt)×100} of from about 62 to about 90; and
(ii) from about 1% to about 10% of a spherical oil absorbing powder having an oil absorbency of at least about 100 ml/100 g;
(b) a volatile silicone oil;
(c) a non-volatile oil;
(d) a solid wax;
(e) a lipophilic surfactant having an HLB of less than about 8; and
(f) water.

2. The solid water-in-oil emulsified make-up composition according to claim 1 wherein the spherical soft focus powder comprises spherical alumina.

3. The solid water-in-oil emulsified make-up composition according to claim 1 wherein the spherical oil absorbing powder comprises spherical silica.

4. The solid water-in-oil emulsified make-up composition according to claim 1 wherein the spherical oil absorbing powder comprises spherical silicone elastomer.

5. The solid water-in-oil emulsified make-up composition according to claim 1 wherein the oil absorbency of the spherical oil absorbing powder is at least about 200 ml/100 g.

6. The solid water-in-oil emulsified make-up composition according to claim 1 wherein the spherical powder has an average particle size of from about 1 μm to about 30 μm.

7. The solid water-in-oil emulsified make-up composition according to claim 1 wherein the lipophilic surfactant comprises an ester-type surfactant and a silicone-type surfactant.

8. The solid water-in-oil emulsified make-up composition according to claim 1 further comprising from about 1% to about 15% of a humectant.

9. The solid water-in-oil emulsified make-up composition according to claim 1 further comprising a film forming polymer.

10. The solid water-in-oil emulsified make-up composition of claim 1 comprising by weight:
(b) from about 20% to about 50% of the volatile silicone oil;
(c) from about 0.5% to about 10% of the non-volatile oil;
(d) from about 1% to about 5% of the solid wax;
(e) from about 1% to about 5% of the lipophilic surfactant; and
(f) water in an amount such that the total of the volatile silicone oil and water is at least about 50%.
